(19) United States
(12) Patent Application Publication
(10) Pub. No.: US 2025/0255525 A1
(43) Pub. Date: Aug. 14, 2025

(54) METHODS AND DEVICES FOR CONTINOUS FATIGUE MONITORING USING SMART DEVICES

(71) Applicant: HUAWEI TECHNOLOGIES CO., LTD., Shenzhen (CN)

(72) Inventors: Yu ZHAO, Kanata (CA); Juntao YE, Kanata (CA); Kuangyu LIU, Shenzhen (CN); Songsong DONG, Shenzhen (CN)

(21) Appl. No.: 19/196,237

(22) Filed: May 1, 2025

Related U.S. Application Data
(63) Continuation of application No. PCT/CN2022/128980, filed on Nov. 1, 2022.

(51) Int. Cl. A61B 5/16; A61B 5/00; A61B 5/024

(52) U.S. Cl. CPC A61B 5/165; A61B 5/02416; A61B 5/7221; A61B 5/7257; A61B 5/7267; A61B 5/746

(57) ABSTRACT

Methods, devices and processor-readable media are described for predicting a fatigue level for a user based on photoplethysmogram (PPG) signals. In various examples, the present disclosure describes a method at a device. A pair of valid PPG snippets are obtained from a continuous PPG signal. A plurality of PPG features are extracted for each valid PPG snippet of the pair of valid PPG snippets. A plurality of pairwise features are then extracted from the plurality of PPG features for each valid PPG snippet and a fatigue level is predicted based on the pairwise features. Optionally, the predicted fatigue level is compared to a pre-defined criteria and a fatigue alert is served to the user based on the comparison. In examples, the disclosed method may help to overcome challenges associated with fatigue prediction in real-life environments where PPG signals can be noisy and impacted by subject variability.

### CROSS-REFERENCE TO RELATED APPLICATIONS

The present disclosure is a continuation of PCT Application No. PCT/CN2022/128980, filed on Nov. 1, 2022, entitled “METHODS AND DEVICES FOR CONTINUOUS FATIGUE MONITORING USING SMART DEVICES”, the disclosure of which is hereby incorporated by reference in its entirety.

### TECHNICAL FIELD

The present disclosure relates to the field of continuous biosignal monitoring using smart devices, in particular systems and methods for monitoring fatigue.

### BACKGROUND

Fatigue may be experienced by an individual as a persistent feeling of physical and/or mental tiredness or weakness. Fatigue may be caused by many factors, including but not limited to: illness, lifestyle, diet, work style and psychology. For example, many pathological conditions such as thyroid disorder, heart disease or diabetes may contribute to fatigue in patients. Lifestyle choices including poor sleep habits (e.g. lack of sleep or oversleeping) may contribute to excessive daytime fatigue. Alcohol use or drug use may stimulate the nervous system and disturb sleeping patterns causing fatigue. In many cases, work conditions or requirements including the need to maintain constant concentration on repetitive tasks may also incur fatigue. For example, long-haul drivers who drive continuously for long periods of time may commonly experience fatigue. Other contributors to fatigue may include long working hours, physical and mental overload, and irregular working hours (e.g. shift work). Finally, individuals experiencing feelings of anxiety, depression or high levels of stress also commonly report chronic fatigue.

Fatigue can negatively an individual's physical or mental performance in a variety of ways. Fatigue may increase the likelihood of an individual being distracted, feeling drowsy, experiencing headaches or dizziness, demonstrating impaired coordination (e.g. hand-to-eye coordination) and impaired decision-making capacity. Fatigued individuals are less efficient and less productive in their tasks. Moreover, fatigue dramatically increases the risks of workplace accidents and injuries, for example as related to driving or other vehicle operation, machine operation, construction or mining, etc. Traffic accidents are a leading cause of fatalities, with estimates of approximately 1.3 million fatalities annually and with fatigue contributing to up to 20% of road accidents.

Therefore, it would be useful to provide a solution that enables continuous fatigue monitoring of individuals in real-world environments.

### SUMMARY

The present disclosure describes devices, methods, and processor-readable media for predicting a user's fatigue level based on photoplethysmogram (PPG) signals. In various examples, the present disclosure describes a method at a device. A pair of valid PPG snippets are obtained from a continuous PPG signal. A plurality of PPG features are extracted for each valid PPG snippet of the pair of valid PPG snippets. A plurality of pairwise features are then extracted from the plurality of PPG features for each valid PPG snippet and a fatigue level is predicted based on the pairwise features. Optionally, the predicted fatigue level is compared to a pre-defined criteria and a fatigue alert is served to the user based on the comparison. In examples, the disclosed method may help to overcome challenges associated with fatigue prediction in real-life environments where PPG signals can be noisy and impacted by subject variability.

The disclosed systems and methods enable fatigue level prediction using valid PPG signals that are not contaminated with motion artifacts (MA) or other noise. This enables improved fatigue prediction performance under real-world conditions where a user may be moving and conducting their daily activities.

In various examples, the present disclosure provides the technical effect that a fatigue level is predicted for a user based on a comparison of inter-user PPG data. Inputs obtained from a PPG sensor are validated to remove motion artifacts (MA) or other noise and PPG features related to a user's heart rate and heart rate variability are inputted into a progressive feature mix to predict a user's fatigue level.

In an example aspect, the present disclosure describes a method for predicting a fatigue level for a user. The method comprises: obtaining a pair of valid PPG snippets from a PPG signal; extracting a plurality of PPG features for each valid PPG snippet of the pair of valid PPG snippets; extracting a plurality of pairwise features from the plurality of PPG features for each valid PPG snippet; and predicting a fatigue level for the user based on the pairwise features.

In the preceding example aspect of the method, wherein obtaining a pair of valid PPG snippets from a PPG signal comprises: extracting a pair of raw PPG snippets from the PPG signal; preprocessing each raw PPG snippet of the pair of raw PPG snippets to generate a pair of processed PPG snippets; and validating each processed PPG snippet of the pair of processed PPG snippets using a trained PPG signal validator to obtain the pair of valid PPG snippets.

In any of the preceding example aspects of the method, wherein extracting a plurality of PPG features for each valid PPG snippet of the pair of valid PPG snippets comprises: detecting a plurality of peaks in each valid PPG snippet of the of the pair of valid PPG snippets; obtaining a plurality of N-N intervals for each valid PPG snippet of the of the pair of valid PPG snippets based on the respective plurality of peaks; and extracting a plurality of PPG features for each valid PPG snippet of the of the pair of valid PPG snippets based on the respective plurality of N-N intervals.

In some example aspects of the method, wherein preprocessing each raw PPG snippet of the pair of raw PPG snippets to generate a pair of processed PPG snippets comprises: filtering each raw PPG snippet of the pair of raw PPG snippets with bandpass filter having a bandpass frequency 0.6-8.0 Hz; and normalizing each raw PPG snippet of the pair of raw PPG snippets to have a mean of zero and a standard deviation of 1.

In some example aspects of the method, the trained PPG signal validator is a fast fourier transform (FFT) based multi-layer perceptron network (MLP).

In some example aspects of the method, the trained PPG signal validator is a temporal convolutional network (TCN).

In any of the preceding example aspects of the method, wherein predicting the fatigue level comprises: inputting the pairwise features into a progressive feature search tree to obtain a predicted fatigue level, the predicted fatigue level including an odds ratio and a probability of experiencing a change in fatigue level.

In the preceding example aspect of the method, wherein prior to inputting the pairwise features into a progressive feature search tree: building a progressive feature search tree by computing one or more relationships between a respective one or more pairwise features and a respective fatigue level.

In the preceding example aspect of the method, wherein the one or more relationships between a respective one or more pairwise features and a respective fatigue level is computed using Fisher's exact test.

In the preceding example aspect of the method, wherein the respective one or more pairwise features correspond to a respective tier level and the progressive feature search tree is built by progressively computing the one or more relationships between the respective one or more pairwise features and the respective fatigue level based on the respective tier level.

In some example aspects of the method, the method further comprises: prior to extracting a plurality of pairwise features from the plurality of PPG features for each valid PPG snippet; classifying the pair of valid PPG snippets using a trained differential network, the classification describing a degree of similarity between the pair of valid PPG snippets; and in response to the pair of valid PPG snippets being classified with a low degree of similarity, extracting the plurality of pairwise features from the plurality of PPG features for each valid PPG snippet in the pair of valid PPG snippets.

In any of the preceding example aspects of the method, the method further comprises: comparing the fatigue level to a pre-defined criteria; and serving a fatigue alert to the user based on the comparison.

In an example aspect, the present disclosure describes a system. The system comprises: a PPG sensor; one or more memories storing executable instructions; and one or more processors coupled to the PPG sensor and one or more memories, the executable instructions configuring the one or more processors to: obtain a pair of valid PPG snippets from a PPG signal; extract a plurality of PPG features for each valid PPG snippet of the pair of valid PPG snippets; extract a plurality of pairwise features from the plurality of PPG features for each valid PPG snippet; and predict a fatigue level for the user based on the pairwise features.

In the preceding example aspect of the system, wherein the executable instructions, when executed by the one or more processors to obtain a pair of valid PPG snippets from a PPG signal, further cause the system to: extract a pair of raw PPG snippets from the PPG signal; preprocess each raw PPG snippet of the pair of raw PPG snippets to generate a pair of processed PPG snippets; and validate each processed PPG snippet of the pair of processed PPG snippets using a trained PPG signal validator to obtain the pair of valid PPG snippets.

In any of the preceding example aspects of the system, wherein the executable instructions, when executed by the one or more processors to extract a plurality of PPG features for each valid PPG snippet of the pair of valid PPG snippets, further cause the system to: detect a plurality of peaks in each valid PPG snippet of the of the pair of valid PPG snippets; obtain a plurality of N-N intervals for each valid PPG snippet of the of the pair of valid PPG snippets based on the respective plurality of peaks; and extract a plurality of PPG features for each valid PPG snippet of the of the pair of valid PPG snippets based on the respective plurality of N-N intervals.

In any of the preceding example aspects of the system, wherein the executable instructions, when executed by the one or more processors to predict the fatigue level, further cause the system to: input the pairwise features into a progressive feature search tree to obtain a predicted fatigue level, the predicted fatigue level including an odds ratio and a probability of experiencing a change in fatigue level.

In the preceding example aspect of the system, wherein the executable instructions, when executed by the one or more processors, further cause the system to: prior to inputting the pairwise features into a progressive feature search tree: build a progressive feature search tree by computing one or more relationships between a respective one or more pairwise features and a respective fatigue level, the one or more relationships between a respective one or more pairwise features and a respective fatigue level being computed using Fisher's exact test.

In a preceding example aspect of the system, wherein the executable instructions, when executed by the one or more processors, further cause the system to: prior to extracting a plurality of pairwise features from the plurality of PPG features for each valid PPG snippet: classify the pair of valid PPG snippets using a trained differential network, the classification describing a degree of similarity between the pair of valid PPG snippets; and in response to the pair of valid PPG snippets being classified with a low degree of similarity, extract the plurality of pairwise features from the plurality of PPG features for each valid PPG snippet in the pair of valid PPG snippets.

In any of the preceding example aspects of the system, wherein the executable instructions, when executed by the one or more processors, further cause the system to: compare the fatigue level to a pre-defined criteria; and serve a fatigue alert to the user based on the comparison.

In some aspects, the present disclosure describes a non-transitory computer-readable medium having machine-executable instructions stored thereon which, when executed by one or more processors of a computing system, cause the computing system to: obtain a pair of valid PPG snippets from a PPG signal; extract a plurality of PPG features for each valid PPG snippet of the pair of valid PPG snippets; extract a plurality of pairwise features from the plurality of PPG features for each valid PPG snippet; and predict a fatigue level for the user based on the pairwise features.

### BRIEF DESCRIPTION OF THE DRAWINGS

Reference will now be made, by way of example, to the accompanying drawings which show example embodiments of the present application, and in which:

FIG. 1 is a block diagram illustrating an example hardware structure of a computing system that may be used for implementing methods to predict a fatigue level, in accordance with examples of the present disclosure;

FIG. 2 is a block diagram illustrating an example architecture of the fatigue prediction system, in accordance with example embodiments of the present disclosure;

FIG. 3 is a block diagram illustrating an example architecture of a PPG preprocessor, in accordance with example embodiments of the present disclosure;

FIG. 4A is a schematic diagram illustrating an example continuous raw PPG signal and raw PPG snippets associated with respective time steps in a series of time steps, in accordance with example embodiments of the present disclosure;

FIG. 4B is a schematic diagram illustrating an example processed PPG snippet and corresponding PPG segments, in accordance with example embodiments of the present disclosure;

FIG. 5 is a block diagram illustrating an example architecture of a PPG Feature Extractor, in accordance with examples of the present disclosure;

FIG. 6 is a block diagram illustrating an example architecture of a predictor, in accordance with examples of the present disclosure;

FIG. 7A-D are schematic diagrams illustrating example heart rate patterns, in accordance with examples of the present disclosure;

FIG. 8 is an example progressive feature search tree that may be used in accordance with examples of the present disclosure; and

FIG. 9 is a schematic diagram illustrating an example continuous raw PPG signal and corresponding PPG snippets forming two consecutive pairs of PPG snippets associated with respective time steps in a series of time steps, in accordance with example embodiments of the present disclosure

FIG. 10 is a flowchart illustrating steps of an example method 1000 for predicting a fatigue level 270, in accordance with examples of the present disclosure.

### DETAILED DESCRIPTION

The following describes example technical solutions of this disclosure with reference to accompanying drawings.

The present disclosure describes devices, methods, and processor-readable media for predicting a user's fatigue level based on photoplethysmogram (PPG) signals. In various examples, the present disclosure describes a method at a device. A pair of valid PPG snippets are obtained from a continuous PPG signal. A plurality of PPG features are extracted for each valid PPG snippet of the pair of valid PPG snippets. A plurality of pairwise features are then extracted from the plurality of PPG features for each valid PPG snippet and a fatigue level is predicted based on the pairwise features. Optionally, the predicted fatigue level is compared to a pre-defined criteria and a fatigue alert is served to the user based on the comparison. In examples, the disclosed method may help to overcome challenges associated with fatigue prediction in real-life environments where PPG signals can be noisy and impacted by subject variability. The disclosed system may also help to overcome challenges associated with fatigue monitoring performance using smart devices, for example, using smart watches.

To assist in understanding the present disclosure, some existing techniques for fatigue monitoring are now discussed.

To mitigate the risk of fatigue, it is important to devise an automatic system that can continuously monitor the levels of fatigue for fatigue management. Existing approaches for fatigue monitoring can be broadly classified into two categories: 1) camera-based approaches and 2) smart watch based approaches.

Camera based approaches detect fatigue using computer vision techniques to extract behavioral signs from video streams, e.g., yawning and eyelid closures. Camera based approaches have been deployed by vehicle manufacturers to detect driver fatigue. However, the usage of cameras in the approaches introduces some limitations: A first limitation includes usage scenarios. Cameras need to be mounted to fixed locations and cannot continuously monitor fatigue when users perform a variety of tasks in different locations. Another limitation includes difficulties with cameras in performing early detection. Commonly, users display physical or behavioral signs of fatigue, such as yawning and eyelid closure, after fatigue has already onset. The display of physical or behavioral signs of fatigue may suggest that a user is already experiencing high levels of fatigue. As such, detection of physical or behavioral signs may occur too late to avoid fatigue related risks. Another limitation includes sensitivity of computer vision techniques to environmental factors. The detection performance of behavioral signs of fatigue can be impacted dramatically by environmental factors, such as light and reflection from eye glasses. Finally, camera-based approaches introduce privacy issues. Users may request to turn off fatigue monitoring systems to resolve privacy concerns.

Smart watch based approaches collect physiological signals from a user's wrist to detect fatigue. The collected physiological signals may include photoplethysmogram (PPG). PPG signals are obtained from optical sensors which consist of a light emitter and detector, where the light emitter continuously emits light to the skin and the detector absorbs the reflected light. As blood volume changes in a cardiac cycle, the amount of absorbed light changes, therefore a peak in PPG signals corresponds to the cardiac cycle. The interval between two peaks reflects the interval between two heartbeats. Therefore, PPG signals can be used to extract heart rate (i.e., HR) and heart rate variability (i.e., HRV). As fatigue induces the changes of HR and HRV, wearable devices can perform early detection of fatigue. Wearable devices enable continuous fatigue monitoring in an unobtrusive manner. Therefore, smart watch based approaches may overcome some of the previously described challenges associated with camera-based approaches.

However, existing smart-watch based fatigue prediction methods were developed using PPG signals collected from controlled environments in laboratory settings. As a result, existing smart-watch based approaches perform poorly on real-world data. The resulting poor performance may be due to a number of reasons: Firstly, PPG signals are noisy. Motion artifacts (MA) may introduce noise in PPG signals, interfering with the estimation of heart rate (HR) and heart rate variability (HRV) and impacting fatigue prediction. Secondly, fatigue is not the only factor to induce changes in PPG signals. PPG signals can be influenced by many other factors, for example, emotion and pathological factors and it may be difficult to discern changes in PPG that are directly caused by fatigue. Finally, HR and HRV vary significantly between individuals, therefore methods to predict fatigue based on HR or HRV information for one individual may not perform well for another individual.

The present disclosure describes examples that may help to address some or all of the above drawbacks of existing technologies.

In the present disclosure, a “fatigue level” can mean: a measure or a ranking of the degree of fatigue experienced by an individual, for example, a ranking based on the Stanford Sleepiness Scale.

In the present disclosure, the “Stanford Sleepiness Scale (SSS)” refers to a self-assessment questionnaire that is used to measure levels of fatigue or sleepiness or alternately, levels of alertness in an individual. Individuals rank their level of fatigue on a scale from 1-7, based on the following descriptions: 1—“Feeling active, vital, alert or wide awake.” 2—“Functioning at high levels, but not at peak; able to concentrate.” 3—“Awake but relaxed; responsive but not fully alert.” 4—“Somewhat foggy, let down.” 5—“Foggy; losing interest in remaining awake; slowed down.” 6—“Sleepy, woozy, fighting sleep; prefer to lie down.” 7—“No longer fighting sleep, sleep onset soon; having dream-like thoughts.”

In the present disclosure, a “cardiac cycle” can mean: the activity of the heart from the beginning of one heartbeat to the start of the next heartbeat. A cardiac cycle consists of two phases: the first phase in which the heart fills with blood (e.g. diastole) and the second phase where the heart contracts and pumps blood through the circulatory system (e.g. systole). A pressure pulse may be detected in the circulatory system associated with the contraction of the various chambers of the heart during each cardiac cycle.

In the present disclosure, a “photoplethysmogram (PPG)” is a plethysmogram that is obtained using optical techniques. In examples, a PPG may be obtained using a pulse oximeter, which measures the change in blood volume circulating in the subcutaneous tissue of an individual by illuminating the skin and measuring changes in light absorption. Due to changes in blood volumes in the skin during each cardiac cycle, a variation may be observed in the amount of light that is detected by a photodiode. In examples, the variation observed with each cardiac cycle may resemble a periodic function, characterized by a peak (and followed by a trough) of varying amplitude and may be sampled and recorded as a time-series PPG signal.

In the present disclosure, “heart rate (HR)” can mean: the number of heartbeats measured for an individual over a duration of time. For example, HR may be represented as the number of heartbeats measured per minute (e.g. beats per min or bpm).

In the present disclosure, “inter-beat interval (IBI)” or “N-N interval” can mean: the duration of time between each heartbeat. IBI is commonly measured as the interval between R peaks (R-R) in a QRS complex of a traditional electrocardiogram (ECG) heart-beat waveform or the interval between peaks in a PPG signal or as the time interval between normalized R peaks (N-N) in a PPG signal.

In the present disclosure, “heart rate variability (HRV) can mean: a measurement of the variation in time intervals between successive heartbeats, or simply the variation in IBI. In examples, HRV may be influenced by multiple factors, including fatigue, emotion, exercise or pathological factors.

In the present disclosure, a “PPG snippet” can mean: a subset of a PPG signal that has been extracted or “snipped” from a larger or a continuous data stream. In some examples, a PPG snippet may be a portion of a raw or processed PPG signal. In some examples, a PPG snippet may have a duration of 5 minutes, or a PPG snippet may have a longer or shorter duration. In some examples, a PPG snippet may be a raw PPG snippet (e.g. a subset of raw PPG signal), a processed PPG snippet (e.g. a subset of processed PPG signal) or a valid PPG snippet (e.g. a subset of processed PPG signal that has been classified as being valid PPG signals).

In the present disclosure, a “pair of PPG snippets” or a “PPG snippet pair” can mean: two PPG snippets corresponding to two distinct time steps in a series of time steps. In examples, each PPG snippet in a PPG snippet pair may include an identifier or a tag to efficiently distinguish one PPG snippet from another. In examples, a PPG snippet pair may be a labeled PPG snippet pair and may be labeled with ground truth fatigue level information for use in model training. In examples, a pair of PPG snippets may be used to evaluate a fatigue level in a user, using, for example, information obtained from each PPG snippet in the pair of PPG snippets including N-N intervals, PPG features or pairwise features, among others.

In the present disclosure, an “N-N interval series” can mean: a collection of N-N intervals extracted from a valid PPG snippet and concatenated into a time series that has the same duration as a PPG snippet (e.g. 5 minutes).

In the present disclosure, a “PPG Feature” can mean: a feature that describes a characteristic of a PPG signal over a specified time duration. For example, a PPG feature may be related to HR (e.g. including, for example, a maximum HR, a minimum HR, a mean HR, among others) or may be related to HRV (including, for example, percentage of N-N interval differences above a threshold, RMS of squared N-N intervals, standard deviation of N-N intervals or standard deviation of an N-N interval series, among others). In the present disclosure, a percentage of N-N interval differences above a threshold can mean: A number of pairs of adjacent N-N intervals differing by more than a specified threshold (e.g. 50 ms) in a series of N-N intervals, with respect to the total number of pairs of adjacent N-N intervals in the series of N-N intervals.

In the present disclosure, a “Pairwise Feature” can mean: a feature that describes a change in a PPG feature between two time steps. For example, pairwise features may include changes in mean HR between time step t and time step t−1, changes in N-N intervals or N-N interval differences between time step t and time step t−1, or changes in minimum and maximum HR between time step t and time step t−1 that visually resemble a defined heart rate pattern, among others.

In the present disclosure, “Fisher's exact test” is a statistical significance test used with categorical data to examine the significance of the association (or contingency) between two categorical variables. In examples, Fisher's test uses contingency tables to summarize the relationship between categorical variables, for example, by displaying frequency distribution of the variables.

Other terms used in the present disclosure may be introduced and defined in the following description.

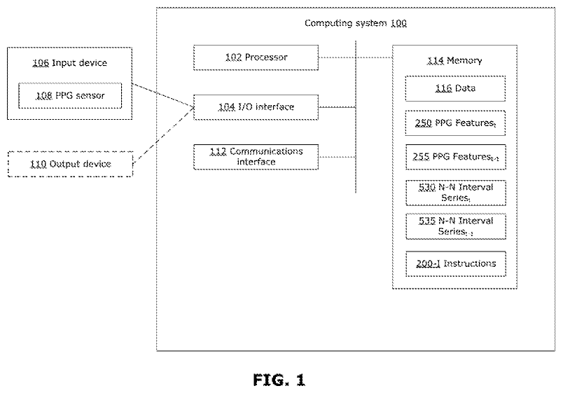

FIG. 1 is a block diagram illustrating an example hardware structure of a computing system 100 that may be used for implementing methods to predict a fatigue level, in accordance with examples of the present disclosure. Examples of the present disclosure may be implemented in other computing systems, which may include components different from those discussed below. The computing system 100 may be used to execute instructions for predicting a fatigue level 270 in a user, using any of the examples described herein. The computing system 100 may also be used to train blocks of the fatigue prediction system 200, or blocks of the fatigue prediction system 200 may be trained by another computing system.

Although FIG. 1 shows a single instance of each component, there may be multiple instances of each component in the computing system 100. Further, although the computing system 100 is illustrated as a single block, the computing system 100 may be a single physical machine or device (e.g., implemented as a single computing device, such as a single workstation, single end user device, single server, etc.), and may include mobile communications devices (smartphones), laptop computers, tablets, desktop computers, vehicle driver assistance systems, wearable devices (e.g. smart watches), assistive technology devices, medical diagnostic devices, virtual reality devices, augmented reality devices and Internet of Things (IoT) devices, among others.

The computing system 100 includes at least one processor 102, such as a central processing unit, a microprocessor, a digital signal processor, an application-specific integrated circuit (ASIC), a field-programmable gate array (FPGA), a dedicated logic circuitry, a dedicated artificial intelligence processor unit, a graphics processing unit (GPU), a tensor processing unit (TPU), a neural processing unit (NPU), a hardware accelerator, or combinations thereof.

The computing system 100 may include an input/output (I/O) interface 104, which may enable interfacing with an input device 106 and/or an optional output device 110. In the example shown, the input device 106 (e.g., a keyboard, a mouse, a microphone, a touchscreen, and/or a keypad) may also include a PPG sensor 108. In the example shown, the optional output device 110 (e.g., a display, a speaker and/or a printer) are shown as optional and external to the computing system 100. In other example embodiments, there may not be any input device 106 and output device 110, in which case the I/O interface 104 may not be needed.

The computing system 100 may include an optional communications interface 112 for wired or wireless communication with other computing systems (e.g., other computing systems in a network). The communications interface 112 may include wired links (e.g., Ethernet cable) and/or wireless links (e.g., one or more antennas) for intra-network and/or inter-network communications.

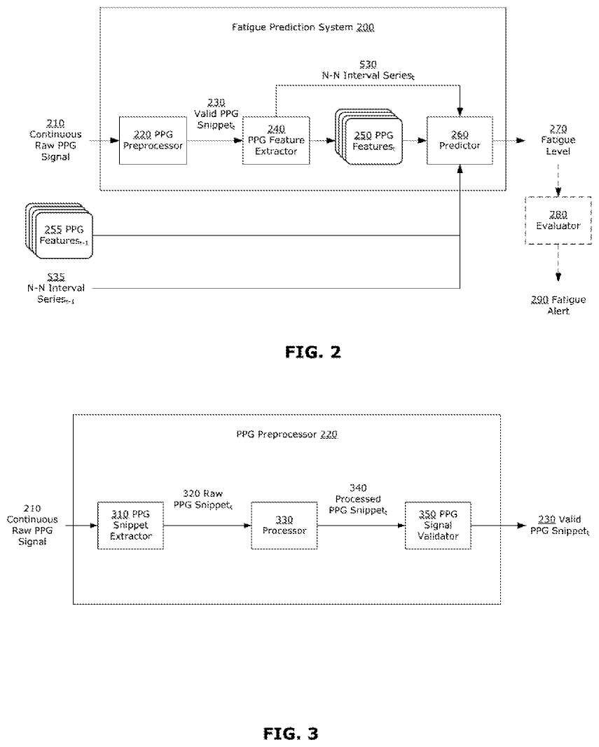

The computing system 100 may include one or more memories 114 (collectively referred to as “memory 114”), which may include a volatile or non-volatile memory (e.g., a flash memory, a random access memory (RAM), and/or a read-only memory (ROM)). The non-transitory memory 114 may store instructions for execution by the processor 102, such as to carry out examples described in the present disclosure. For example, the memory 114 may store instructions for implementing any of the networks and methods disclosed herein. The memory 114 may include other software instructions, such as for implementing an operating system (OS) and other applications/functions. The instructions can include instructions 200-I for implementing and operating the fatigue prediction system 200 described below with reference to FIG. 2.

The memory 114 may also store other data 116, information, rules, policies, and machine-executable instructions described herein, including PPG features such as PPG features: 250 for a current time step t, or PPG featuresₜ₋₁ 255 for a previous time step t−1, which may have been generated by the fatigue prediction system 200 and stored in the memory 114. The memory 114 may also store data including an N-N interval series, 530 for a current time step t and an N-N interval seriesₜ₋₁ 535 for a previous time step t−1, which may have been generated by the fatigue prediction system 200.

In some examples, the computing system 100 may also include one or more electronic storage units (not shown), such as a solid state drive, a hard disk drive, a magnetic disk drive and/or an optical disk drive. In some examples, data and/or instructions may be provided by an external memory (e.g., an external drive in wired or wireless communication with the computing system 100) or may be provided by a transitory or non-transitory computer-readable medium. Examples of non-transitory computer readable media include a RAM, a ROM, an erasable programmable ROM (EPROM), an electrically erasable programmable ROM (EEPROM), a flash memory, a CD-ROM, or other portable memory storage. The storage units and/or external memory may be used in conjunction with memory 114 to implement data storage, retrieval, and caching functions of the computing system 100. The components of the computing system 100 may communicate with each other via a bus, for example.

FIG. 2 is a block diagram illustrating an example architecture of the fatigue prediction system 200, in accordance with example embodiments of the present disclosure.

In some examples, the fatigue prediction system 200 may receive a raw continuous PPG signal 210 and outputs a predicted fatigue level 270 for a user. In examples, a continuous biological signal such as a PPG signal may be monitored for an individual using an electronic device, for example, a smart watch secured to an individual's wrist and in contact with the individual's skin. In examples, the smart watch may include a PPG sensor 108 (e.g. a pulse oximeter), for measuring the raw continuous PPG signal 210. In examples, in obtaining the raw continuous PPG signal 210, the PPG sensor 108 may emit light in proximity to the user's skin and may detect absorbed or reflected light from skin. In examples, the recorded raw PPG signal representing variations in blood volume within subcutaneous tissue may be observed to vary in intensity with each heartbeat.

Although methods and devices related to the use of a smart watch are described herein, it should be understood that other wearable electronic devices may be used to monitor and record PPG signals for an individual.

In examples, a PPG preprocessor 220 of the fatigue prediction system 200 may receive the raw continuous PPG signal 210 and may output a valid PPG snippet, 230 corresponding to a respective time step t, as described below with respect to FIG. 3. In examples, a valid PPG snippet, 230 may be a pre-processed PPG signal of a pre-defined duration that can be used to predict fatigue in a user.

FIG. 3 is a block diagram illustrating an example architecture of a PPG preprocessor 220, in accordance with example embodiments of the present disclosure. In examples, a PPG snippet extractor 310 of the PPG preprocessor 220 receives the continuous raw PPG signal 210 and extracts a raw PPG snippet, 320 corresponding to a respective time step t. In examples, the raw PPG snippet, 320 may represent a portion of the continuous raw PPG signal 210 having a pre-determined duration (e.g. a duration of 5 minutes) that has been extracted or “snipped” from the raw continuous PPG signal 210 at a respective time step t.

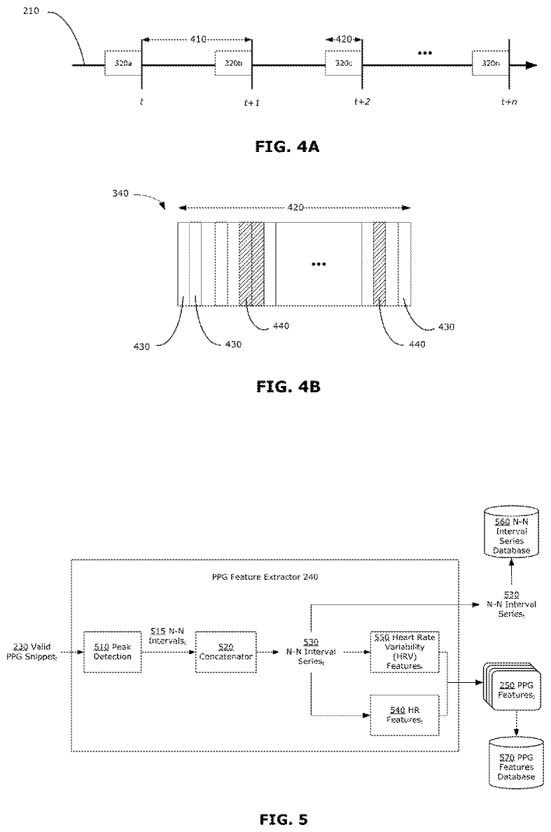

FIG. 4A is a schematic diagram illustrating an example continuous raw PPG signal 210 and raw PPG snippets 320 associated with respective time steps t in a series of time steps, in accordance with example embodiments of the present disclosure. In examples, raw PPG snippets 320a, 320b, 320c . . . 320n may be recorded at regular time-step intervals 410 corresponding to respective time steps t, t+1, t+2 . . . t+n, respectively. In examples, each raw PPG snippet 320 may have a pre-defined snippet duration 420 corresponding to a respective time step t. In some embodiments, for example, the time-step interval 410 may be 15 minutes and the pre-defined snippet duration 420 may be 5 minutes.

Returning to FIG. 3, the raw PPG snippet, 320 may be input into a processor 330 to generate a processed PPG snippet, 340. It is understood that PPG signals can be noisy, for example, due to motion artifacts (MA) caused for example, by ambient light leaking into the gap between a PPG sensor 108 and the surface of a user's skin. In this regard, a raw PPG snippet, 320 may include a plurality of valid PPG signals having clear peaks and a plurality of invalid PPG signals contaminated with motion artifacts. In examples, the processor 330 may process the raw PPG snippet, 320 to help remove some noise associated with MA, among other sources of noise in the signal. In examples, the processor 330 may segment the raw PPG snippet, 320 into a plurality of non-overlapping PPG segments, each having a pre-determined segment duration. In some embodiments, for example, the raw PPG snippet, 320 may be segmented into 60 non-overlapping PPG segments, each having a duration of 5 seconds. In examples, the processor 330 may process each 5-second PPG segment of the raw PPG snippet, 320, for example, using a bandpass filter with a bandpass frequency of 0.6-8.0 Hz. In examples, the processor 330 may then normalize each filtered 5-second PPG segment of the raw PPG snippet, 320 to have a mean value of 0 and a standard deviation of 1.

In examples, the processed PPG snippet, 340 may be input to a PPG signal validator 350 to classify each 5-second PPG segment of the processed PPG snippet, 340 as either valid or invalid and to generate a valid PPG snippet, 230 for use in fatigue prediction.

FIG. 4B is a schematic diagram illustrating an example processed PPG snippet, 340 and corresponding PPG segments, in accordance with example embodiments of the present disclosure. In examples, each PPG segment may be classified as a valid PPG segment 430 or an invalid PPG segment 440, where a PPG segment may be considered to be a valid PPG segment 430 if it meets a set of criteria for classifying a PPG segment as valid. In examples, criteria for classifying a PPG segment as a valid PPG segment 430 may include: 1) having clear peaks in the PPG segment signal and 2) the duration between peaks are consistent with a typical heart rate (i.e., 0.5 Hz-3 Hz), among others. In examples, an invalid PPG segment 440 may represent a PPG signal that has been contaminated by MA and may exhibit no visible peaks that follow typical heart rate frequencies, among others. In some embodiments, for example, only valid PPG signals may be used to predict fatigue while invalid PPG signals may be discarded. In some embodiments, for example, the PPG signal validator 350 may generate a valid PPG snippet, 230 from a processed PPG snippet, 340 by concatenating the valid PPG segments 430 and discarding the invalid PPG segments 440. For example, the valid PPG snippet, 230 may be shorter in duration than the processed PPG snippet, 340, depending on the number of invalid PPG segments 440 which were discarded.

In examples, the PPG signal validator 350 may be a trained machine learning model, for example, a signal classifier. In examples, the PPG signal validator 350 may be trained by supervised machine learning using a labeled dataset. In examples, in assembling a training dataset, a collection of 35,800 PPG segments (e.g. 5-second PPG segments) were manually examined and labeled as “valid” or “invalid” based on the quality of the segment. For example, of the 35,800 labelled PPG segments, 15,800 were labelled as “valid” while the remaining 20,000 segments were labelled as “invalid”.

In one embodiment, for example, the PPG signal validator 350 may be a Fourier transform based multi-layer perception network (FFT). In examples, the FFT approach uses fast Fourier transform to convert a PPG segment from the time domain to the frequency domain, and derive power of frequencies of the PPG segment. In examples, the power of frequencies may then be fed into a trained multi-layer perceptron network for signal classification as a valid PPG segment 430 or an invalid PPG segment 440.

In another embodiment, for example, the PPG signal validator 350 may be a temporal convolutional network (TCN). In examples, the TCN approach may input each PPG segment of a processed PPG snippet 340 into a trained temporal convolutional network for signal classification as a valid PPG segment 430 or an invalid PPG segment 440.

In examples, both the FFT and TCN classification methods demonstrate good performance in accurately predicting valid PPG segments 430. Both the FFT and TCN approaches classify a 5-second sample in less than 0.1 ms. While the TCN approach demonstrates a slightly higher accuracy compared to the FFT approach, the FFT approach is considerably more efficient since a PPG snippet in the frequency domain has lower dimensions compared to the PPG snippet in the time domain.

Returning to FIG. 2, the valid PPG snippet, 230 may be input to a PPG feature extractor 240 to obtain a set of PPG features, 250 for a current time step t, as described below with respect to FIG. 5.

FIG. 5 is a block diagram illustrating an example architecture of a PPG Feature Extractor 240, in accordance with examples of the present disclosure. In examples, a valid PPG snippet, 230 may be input to the PPG Feature Extractor 240 and peak detection 510 may be performed on the valid PPG snippet, 230 to identify peaks in each 5-second valid PPG segment 430 of the valid PPG snippet, 230. In some embodiments, for example, peaks may be detected by finding local maxima within the signal via comparison to neighbor values, or another method may be used. An example of a peak detection algorithm that can be implemented in example embodiments is described in: Elgendi, Mohamed, et al. “Systolic peak detection in acceleration photoplethysmograms measured from emergency responders in tropical conditions,” PloS one 8.10 (2013): e76585, the entirety of which is hereby incorporated by reference. Another example of a peak detection algorithm that can be implemented in example embodiments is described in: Kazemi, Kianoosh, et al. “Robust PPG Peak Detection Using Dilated Convolutional Neural Networks” Sensors 22.16 (2022): 6054, the entirety of which is hereby incorporated by reference.

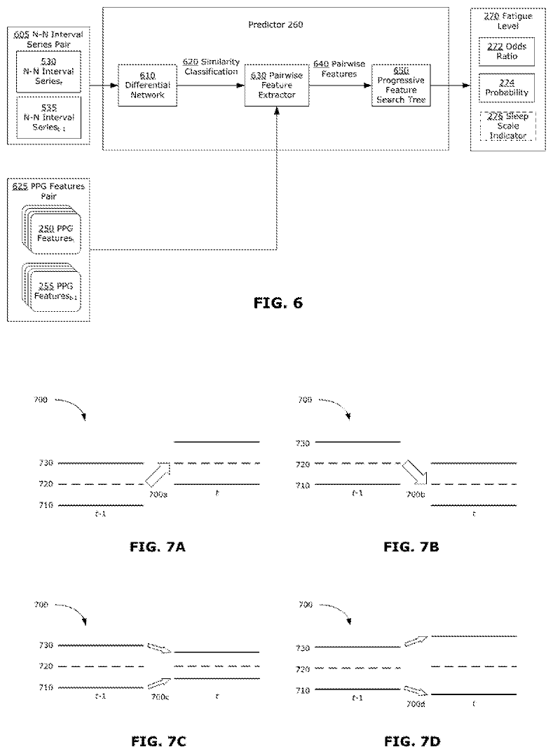

In examples, a sequence of N-N intervals 515 may be extracted for each 5-second valid PPG segment 430 in the valid PPG snippet, 230 based on the detected peaks, where an N-N interval represents the time duration between successive peaks. In examples, the sequence of N-N intervals 515 for each valid PPG segment 430 may then be input to a concatenator 520 to generate an N-N interval seriesₜ 530 corresponding to a time step t, comprising a series of concatenated N-N intervals representative of a pre-defined snippet duration 420 (e.g. 5 minutes in duration). To demonstrate the actions of the concatenator 520, an example is now described. Supposing the valid PPG snippet, 230 included 30 valid PPG segments 430, each having a duration of 5 seconds. In this regard, the valid PPG snippet, 230 may only have a duration of 2.5 minutes, which is shorter than a pre-defined snippet duration 420 of 5 minutes. Therefore, the concatenator 520 may concatenate the sequence of N-N intervals 515 corresponding to the 30 valid PPG segments 430 and may continue appending N-N intervals 515 corresponding to the valid PPG segments 430 in order, until an N-N interval series: 530 is generated that corresponds to the pre-defined snippet duration 420 (e.g. 5 minutes). The N-N interval series: 530 may be output for use by the predictor 260, as described with respect to FIG. 6 below. Optionally, the N-N interval series, 530 may be stored in an N-N interval series database 560 for retrieval, as needed, during prediction operations.

In examples, a set of heart rate (HR) features, 540 may be generated based on the N-N interval seriesₜ 530. In examples, heart rate may be calculated for each N-N interval 515 in the N-N interval series 530 using the following equation:

$$H R = \frac{1}{duration of N - N interval} \qquad ( 1 )$$

where a duration of N-N interval represents a duration in seconds.

In examples, the HR features 540 may include: the maximum HR over a 5-minute duration (Max HR), the minimum HR over a 5 minute duration (Min HR) and the mean HR over a 5-minute duration (Mean HR), among others.

In examples, a set of heart rate variability (HRV) features: 560 may also be generated based on the N-N interval seriesₜ 530. In examples, the HRV featuresₜ 550 may include: the percentage of N-N interval differences greater than 20 ms (PNN20), the percentage of N-N interval differences greater than 50 ms (PNN50), the root mean of squared N-N interval differences (RMSSD), the standard deviation of N-N interval differences (SDSD) and the standard deviation of an N-N interval series (SDNN), among others.

In examples, the HR features: 540 and HRV features: 550 may be combined to form a set of PPG features, 250 corresponding to a time step t. Optionally, the set of PPG featuresₜ 250 may be stored in a PPG features database 570 for retrieval, as needed, during prediction operations.

FIG. 6 is a block diagram illustrating an example architecture of a predictor 260, in accordance with examples of the present disclosure. In examples, the predictor 260 uses a PPG features pair 625 obtained at two different time steps in a series of time steps to infer changes in HR and HRV characteristics that may be indicative of a change in fatigue level 270. In examples, the PPG features pair 625 may include PPG features, 250 associated with a current time step t, and PPG featuresₜ₋₁ 255 associated with a previous time step t−1.

In examples, to effectively infer changes in fatigue level 270 from the PPG features pair 625, the PPG signals obtained for the user at two different time steps in a series of time steps must exhibit significant differences. To confirm that the PPG features pair 625 exhibits significant differences, a differential network 610 may receive an N-N interval series pair 605 corresponding to the PPG features pair 625 and may output a similarity classification 620, where the similarity classification 620 describes a degree of similarity between the N-N interval series pair 605. In examples, the N-N interval series pair 605 may include an N-N interval seriesₜ 530 associated with a current time step t and an N-N interval seriesₜ₋₁ 535 associated with a previous time step t−1. In some embodiments, for example, HR and HRV may be extracted from the N-N interval series 530 and N-N interval seriesₜ₋₁ 535, respectively, and input into two identical neural networks to learn hidden representations. In some embodiments, for example, the two identical neural networks may each be a trained multilayer perceptron (MLP) network. In examples, the output of the two MLP networks may be compared using a contrastive loss 620. An example of a contrastive loss approach that can be implemented in example embodiments is described in: Koch. Gregory, et al . . . “Siamese neural networks for one-shot image recognition.” ICML deep learning workshop, Vol. 2. 2015, the entirety of which is hereby incorporated by reference. In examples, the similarity classification 620 may indicate whether an N-N interval series pair 605 is “similar” or whether the N-N interval series pair 605 is “different”, where an N-N interval series pair 605 that is “different” may be used by the predictor 260 to predict a change in fatigue level 270 for the user.

In examples, the differential network 610 may include two trained machine learning models, for example, two MLP networks. In examples, the differential network 610 may be trained by supervised machine learning using a labeled training dataset. In examples, in assembling a labeled training dataset, a user may be asked to complete a fatigue level self-assessment (e.g. according to the Stanford Sleepiness Scale) at each time step t in the series of time steps and for every time step t thereafter, to serve as a ground truth fatigue level information. In examples, the Stanford Sleepiness Scale uses a ranking on a scale of 1-7 corresponding to a user's feelings of fatigue. In examples, a score of 1 represents a user that is feeling active, vital, alert or wide awake while a score of 7 corresponds to a user no longer fighting sleep, sleep onset soon or having dream-like thoughts. Using this approach, a training dataset was created, including a collection of 4540 pairs of labeled N-N interval series from two different time steps, each pair of labeled N-N interval series labeled with corresponding ground truth fatigue level information. Further, a subset of 607 pairs of labeled N-N interval series from two different time steps were randomly selected from the training dataset and manually labeled as “similar” or “different” based on three factors: 1) observed differences in HR, 2) observed differences in HRV and 3) change in reported fatigue level by the user.

In examples, to train the differential network 610, HR and HRV were extracted separately from each pair of labeled N-N interval series of the subset of 607 pairs of labeled N-N interval series and fed into a respective MLP network of the differential network 610. The output of the two identical neural networks were compared using a contrastive loss function in order to maximize the difference of learned hidden representations for a pair of labeled N-N interval series labeled as “different” and to minimize the difference of learned hidden representations for a pair of labeled N-N snippets labeled as “similar”.

In examples, once the PPG features pair 625 has been confirmed to demonstrate significant differences, a pairwise feature extractor 630 of the predictor 260 may receive the PPG features pair 625, including PPG features, 250 associated with a current time step t in a series of time steps, as well as PPG featuresₜ₋₁ 255 associated with a previous time step t−1, and may extract relevant pairwise features 640. In examples, pairwise features 640 may include features describing changes in values of PPG features between the two time steps, for example, changes in mean HR between time step t and time step t−1, changes in N-N intervals or N-N interval differences, or changes in minimum and maximum HR that visually resemble a defined heart rate pattern 700, as described below with reference to FIGS. 7A-7D. In examples, a pairwise feature 640 may demonstrate an increase in value between two time steps or a decrease in value between two time steps. In examples, an increase in a value of a pairwise feature 640 between two time steps may be described by an “UP” pattern descriptor and a decrease in a value of a pairwise feature 640 between two time steps may be described by a “DOWN” pattern descriptor. In examples, the importance of pairwise features 640 may also be represented by classification in one of a number of tiers, where a tier 1 pairwise feature 640 may be very important in the prediction of fatigue level 270 while a tier 5 pairwise feature 640 may be less important in the prediction of fatigue level 270. A summary of example extracted pairwise features 640 and associated tiers is provided in Table 1 below.

TABLE 1
Example extracted pairwise features 640,
pattern descriptors and assigned tiers

| Pairwise | | Pattern | | Cate- |
|---|---|---|---|---|
| Feature | Description | Descriptor | Tier | gory |
| HRP | Heart Rate Pattern | Up, Down, | 1 | HR |
| | | Shrink, | | |
| | | Enlarge | | |
| HR | Mean Heart Rate | Up, Down | 2 | HR |
| PNN20 | Percentage of N-N interval | Up, Down | 3 | HRV |
| | differences greater than 20 ms | | | |
| PNN50 | Percentage of N-N interval | Up, Down | 3 | HRV |
| | differences greater than 50 ms | | | |
| RMSSD | Root mean of squared N-N | Up, Down | 4 | HRV |
| | interval differences | | | |
| SDSD | Standard deviation of N-N | Up, Down | 4 | HRV |
| | interval differences. | | | |
| SDNN | Standard Deviation of a N-N | Up, Down | 5 | HRV |
| | interval series | | | |

FIG. 7A-D are schematic diagrams illustrating example heart rate patterns 700, in accordance with examples of the present disclosure. In some embodiments, for example, heart rate patterns 700 may describe a relationship between HR features 540 such as differences in minimum HR 710, mean HR 720 and maximum HR 730 between two time steps in a series of time steps, for example, a current time step t and a previous time step t−1.

FIG. 7A illustrates an “UP” heart rate pattern 700a, in which the minimum HR 710 and the maximum HR 730 both increase between time step t−1 and time step t. FIG. 7B illustrates a “DOWN” heart rate pattern 700b, in which the minimum HR 710 and the maximum HR 730 both decrease between time step t−1 and time step t. FIG. 7C illustrates a “SHRINK” heart rate pattern 700c, in which the minimum HR 710 increases and the maximum HR 730 decreases between time step t−1 and time step t. Finally, FIG. 7D illustrates a “ENLARGE” heart rate pattern 700d, in which the minimum HR 710 decreases and the maximum HR 730 increases between time step t−1 and time step t.

Returning to FIG. 6, the pairwise features 640 may be progressively combined using a progressive feature search tree 650 in order to predict a fatigue level 270. In examples, the progressive feature search tree 650 is built by deriving the relationships between progressive tiers of pairwise features 640 and fatigue level 270 using Fisher's exact test. An example of Fisher's test that can be implemented in example embodiments is described in: Fisher, Ronald A, “On the interpretation of χ2 from contingency tables, and the calculation of P,” Journal of the royal statistical society 85.1 (1922): 87-94, the entirety of which is hereby incorporated by reference.

In examples, to derive the relationships between a given pairwise feature 640 and fatigue level 270, a dataset including labeled pairwise features 640 for a collection of corresponding PPG snippet pairs labeled with corresponding ground truth fatigue level information (e.g. fatigue level self-assessments corresponding to two associated time steps) is obtained. The PPG snippet pairs are separated into two groups: 1) a control group and 2) an experimental group. For a particular value of the given pairwise feature 640 (e.g. “UP” heart rate pattern), the control group contains labeled PPG snippet pairs exhibiting the value of the given pairwise feature 640, and the experimental group contains all remaining PPG snippet pairs in the dataset. The following null hypothesis H₀ is tested for the value of the given pairwise feature 640: “The proportion of PPG snippet pairs that experiences increasing fatigue levels in the experimental group and the control group has no difference.”

In examples, the null hypothesis H₀ is evaluated using the Fisher's exact test with 99% confidence level (e.g. p-value <0.01). In examples, an odds ratio (OR) is computed to measure the likelihood of experiencing increasing fatigue levels in the experimental group. If OR=1, the likelihood of experiencing an increase or decrease in fatigue level is equally likely in each group. If OR>1, the PPG snippet pairs in the experimental group are more likely to experience increasing fatigue levels, while if OR<1, the PPG snippet pairs in the experimental group are less likely to experience increasing fatigue levels.

The use of Fisher's exact test is now demonstrated to derive the relationships between a given pairwise feature 640 (e.g. the “UP” heart rate pattern) and fatigue level 270. In examples, a dataset including labeled pairwise features 640 for a collection of PPG snippet pairs labeled with corresponding ground truth fatigue level information is obtained and the PPG snippet pairs are separated into a control group and an experimental group based on the value of the given pairwise feature 640. In examples, the number of instances of increasing fatigue levels and decreasing levels in each group are calculated, as shown in Table 2 below.

TABLE 2
Contingency table for “UP” heart rate pattern

| | Increasing fatigue | Decreasing fatigue |
|---|---|---|
| | levels | levels |
| “UP” heart rate pattern (Control) | 159 | 77 |
| Others (Experimental) | 463 | 627 |

In examples, using the Fisher's exact test, the odds ratio for the “UP” heart rate pattern is 2.8 with a significant p-value (p-value=4.7e−12<0.01). The result indicates that when pairs of PPG snippets exhibit the “UP” heart rate pattern, individuals are 2.8 times more likely to experience a decrease in fatigue level, and have a probability of 67% of experiencing a change in fatigue level (e.g. a decrease in fatigue level).

In examples, the above described approach may be repeated for each value of the tier 1 pairwise feature, to further derive relationships between the pairwise feature 640 and fatigue level 270. For example, a contingency table may be generated for remaining values of the heart rate pattern feature, including the “DOWN” heart rate pattern, the “SHRINK” heart rate pattern and the “ENLARGE” heart rate pattern, and the odds ratio and probability of experiencing a change in fatigue level may be calculated for each value, respectively. In examples, additional pairwise features 640 (e.g. tier 2 pairwise features) may be progressively combined with the tier 1 features and relationships between the combined features and fatigue level may be derived using the Fisher's exact test. A second example is provided to demonstrate the derivation of the relationship between the selected tier 1 pairwise feature 640 of “UP” heart rate pattern and the selected tier 2 pairwise feature 640 of “UP” mean heart rate, and fatigue level 270. In examples, the dataset including a collection of PPG snippet pairs labeled with corresponding ground truth fatigue level information is obtained and the PPG snippet pairs are separated into a control group and an experimental group. In examples, the number of instances of increasing fatigue levels and decreasing levels in each group are calculated, as shown in Table 3 below. In examples, using the Fisher's exact test, the odds ratio for the tier 1 “UP” heart rate pattern feature combined with the tier 2 “UP” mean heart rate feature may be calculated along with the probability of experiencing a change in fatigue level.

TABLE 3
Contingency table for “UP” heart rate pattern
combined with “UP” mean heart rate

| | Increasing | Decreasing |
|---|---|---|
| | fatigue | fatigue |
| | levels | levels |
| “UP” heart rate pattern + “UP” mean | 146 | 72 |
| heart rate (Control) | | |
| Others (Experimental) | 476 | 632 |

In examples, the above described approach may be repeated for each value of each pairwise feature 640, progressing in order based on tier, for example, adding additional tier 2 pairwise features 640, followed tier 3 pairwise features 640 and so on, progressing through all tiers of pairwise features 640 until all combinations are accounted for. In this regard, in performing Fisher's exact test on all progressive combinations of features, a progressive feature search tree 650 is built to query prediction results for a given PPG snippet pair.

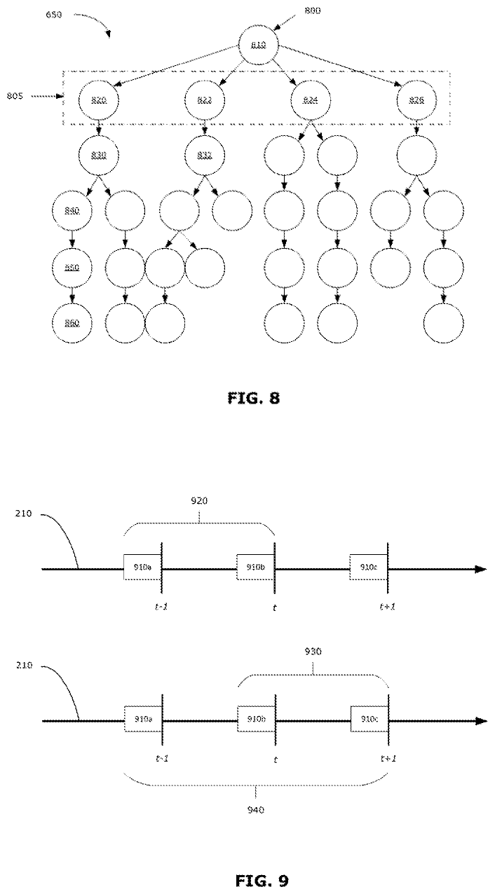

FIG. 8 is an example progressive feature search tree 650 that may be used in accordance with examples of the present disclosure. In examples, the progressive feature search tree 650 comprises multiple nodes 800 (e.g. denoted by circles), arranged in multiple layers 805, where each layer corresponds to a separate tier of pairwise features 640 as presented in Table 1. Each node represents a progressive pairwise feature 640 combination, and includes an associated OR and a probability of a user experiencing a change in fatigue level. In constructing the progressive feature search tree 650, a maximum depth of layers is reached when adding more tiers of features does not produce significant associations with changes in fatigue level 270. In examples, the progressive feature search tree 650 is built to query prediction results for a pair of PPG snippets. To predict fatigue level 270 based on a pair of PPG snippets, pairwise features 640 of the pair of PPG snippets are progressively input to the search tree 650, in order, based on the associated tiers, to a maximum layer depth, and for combinations of pairwise features 640 exhibiting significant associations with fatigue, a corresponding OR and probability of experiencing a change in fatigue level 270 for the feature combination is obtained. In examples, if all of the feature combinations in a pair of PPG snippets are not significantly associated with a change in fatigue level, a prediction cannot be made for the sample.

In examples, the progressive feature search tree 650 begins at node 810. Node 810 includes four branches in the first layer of the search tree based on the tier 1 pairwise feature of “heart rate pattern.” In examples, nodes 820, 822, 824 and 826 may correspond to values of the heart rate pattern including “UP”, “DOWN”, “SHRINK” and “ENLARGE”, respectively. In examples, a next layer corresponding to the tier 2 pairwise feature “mean heart rate” may be added, denoted for example by nodes 830 and 832. In examples, node 830 may correspond to an “UP” mean heart rate and node 832 may correspond to a “DOWN” mean heart rate. In examples, the progressive feature search tree 650 may include further layers, for example as indicated by nodes 840, 850 and 860. In some embodiments, for example, nodes 840, 850 and 860 may correspond to values associated with tier 3, 4 and 5 pairwise features, respectively.

Returning to FIG. 6, a fatigue level 270 may be predicted at a first time step t in a series of time steps and may be repeated for every time step 1 thereafter. In examples, each fatigue level 270 prediction may include an odds ratio 272 and a probability of change in fatigue levels 274 that are obtained from the progressive feature search tree 650. Optionally, the fatigue level 270 prediction may also include a sleep scale indicator 276 that may indicate whether the user's fatigue level 270 with respect to the Stanford Sleepiness Scale is trending upwards, downwards or remaining the same.

Returning to FIG. 2, once a fatigue level 270 has been predicted, an evaluator block 280 may optionally evaluate the fatigue level 270 prediction and determine a risk level associated with the fatigue level 270. In examples, the evaluator may compare the fatigue level 270 to a pre-defined criteria, for example, a threshold value. In other examples, the evaluator may evaluate a trend in fatigue level 270 and may compare the trend to a pre-defined criteria, for example, a value of a slope or another threshold. In examples, the evaluator 280 may serve a fatigue alert 290 to a user based on the comparison, for example, to call attention to a predicted fatigue level 270. In one example scenario, a user may demonstrate an increase in fatigue level 270 while working, for example, while driving or operating machinery. Accordingly, a first fatigue alert 290a may serve an alert or notification to the user calling attention to the increased fatigue or drowsiness, or prompting the user to pause and take a rest, or to take an action such as initiating movement, or getting a coffee, etc. In another example scenario, in response to taking a break or engaging in some action after receiving a first fatigue alert 290a, a user may demonstrate decreasing fatigue levels (e.g. increased alertness). Accordingly, a second fatigue alert 290b may be served to the user indicating that they can return to work. In another example scenario, a user may demonstrate decreasing fatigue levels (e.g. increased alertness) during a time when the user should not be alert, for example, late at night when they should be asleep. Accordingly, a fatigue alert 290 may serve an alert or notification to the user calling attention to the increased alertness, or prompting the user to go to bed.

In another embodiment of the present disclosure, fatigue level 270 prediction may be performed using two or more consecutive pairs of PPG snippets, instead of one pair of PPG snippets. In examples, a first pair of PPG snippets corresponding to time steps t and t−1 in a series of time steps may be used to generate a fatigue level prediction. In examples, a second pair of PPG snippets corresponding to time step t+1 and t may be used to further refine or improve the fatigue level prediction, for example, by examining the changes in HR and HRV between the two or more consecutive pairs of PPG snippets, as described with respect to FIG. 9 below.

FIG. 9 is a schematic diagram illustrating an example continuous raw PPG signal 210 and corresponding PPG snippets 910 forming two consecutive pairs of PPG snippets 940 for use in the prediction of a user's fatigue level 270, in accordance with example embodiments of the present disclosure. In examples, capturing changes in HR and HRV across two consecutive pairs of PPG snippets 940 may provide an improved predicted fatigue level 270 for a user. In examples, two consecutive pairs of PPG snippets 940 may include a first pair of PPG snippets 920 and a second pair of PPG snippets 930.

In examples, the first pair of PPG snippets 920 including PPG snippets 910a and 910b corresponding to time steps t−1 and t, respectively, in a series of time steps, and the second pair of PPG snippets 930 including PPG snippets 910b and 910c corresponding to time step t and t+1, respectively, in the series of time steps may both be obtained. In examples, pairwise features 640 may be generated for each pair of PPG snippets 920 and 930, for example, as previously described with respect to FIG. 6.

In examples, a progressive feature search tree 650 may be constructed for the consecutive pairs of PPG snippets 940, using for example, the method previously described with respect to FIG. 8, starting from tier 1 pairwise features 640 (e.g. heart rate pattern) and progressing to tier 2 features, and so on. In examples, each value combination of the pairwise features 640 across two consecutive pairs of PPG snippets 940 is examined with the Fisher's exact test to evaluate a degree of association with fatigue level 270. In examples, Fisher's exact test is performed on progressive combinations pairwise features 640, in order by tier, for the consecutive pairs of PPG snippets 940 to build the progressive feature search tree 650. In examples, fatigue level 270 may then be predicted for the two consecutive pairs of PPG snippets 940 using the progressive feature search tree 650 as previously described with respect to FIG. 6

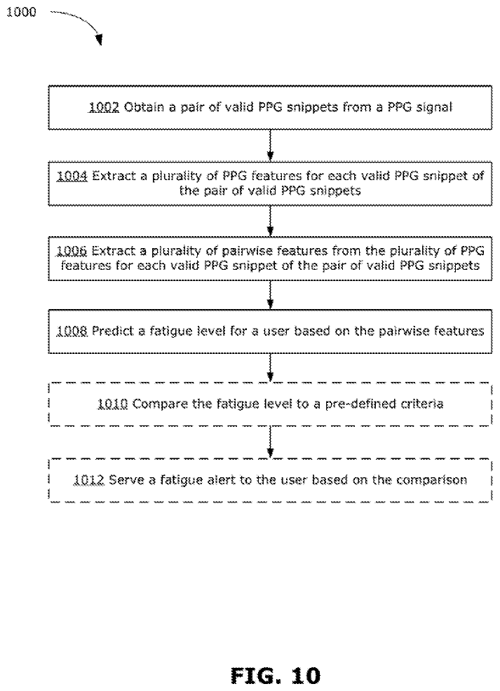

FIG. 10 is a flowchart illustrating steps of an example method 1000 for predicting a fatigue level 270, in accordance with examples of the present disclosure. The method 1000 can be performed by the computing system 100. For example, the processor 102 can execute computer readable instructions (which can be stored in the memory 114) to cause the computing system 100 to perform the method 1000.

Method 1000 begins with step 1002 in which a pair of valid PPG snippets 230 are obtained from a continuous raw PPG signal 210. In examples, in generating the pair of valid PPG snippets, a pair of raw PPG snippets are extracted from the continuous raw PPG signal 210 and preprocessed, for example, to remove motion artifacts or other noise. In examples, the pair of processed PPG snippets may be input to a trained PPG signal validator 350 to generate the pair of valid PPG snippets.

At step 1004, a plurality of PPG features may be extracted for each valid snippet 230 of the pair of valid PPG snippets. In examples, peak detection 510 may be performed on each valid PPG snippet 230 of the of the pair of valid PPG snippets to detect a plurality of peaks in each valid PPG snippet 230. In examples, a plurality of N-N intervals 515 may be obtained for each valid PPG snippet 230 of the pair of valid PPG snippets based on the respective plurality of peaks. In examples, the plurality of PPG features may be extracted for each valid PPG snippet 230 of the pair of valid PPG snippets based on the respective plurality of N-N intervals 515.

At step 1006, a plurality of pairwise features 640 may be extracted from the plurality of PPG features for each valid PPG snippet 230 of the pair of valid PPG snippets.

At step 1008, a fatigue level 270 may be predicted based on the pairwise features 640. In examples, the pairwise features 640 may be input to a progressive feature search tree 650 to obtain a fatigue level prediction 270. In examples, the pairwise features 640 may be progressively input to the progressive feature search tree 650, in order, based on the associated tier for each pairwise feature 640, where tier 1 pairwise features 640 are input first, followed by tier 2 pairwise features 640, and so on, to a maximum layer depth. In examples, if a node in the progressive search tree 650 determines that the progressive pairwise features 640 have significant associations with fatigue, an odds ratio 272 and a probability of experiencing a change in fatigue level 274 may be displayed.

Optionally, at step 1010, the fatigue level 270 may be compared to a pre-defined criteria and at step 1012, a fatigue alert 290 may be served to a user based on the comparison.

In some examples, the fatigue level 270 may be output to an application on an electronic device (e.g., a software application executed by the computing system 100) to serve a fatigue alert 290 to the individual. In other examples, the fatigue level 270 may be output to an application to be executed by an in-vehicle computing system or a heavy machinery operating system, to serve a fatigue alert 290 individual operating the vehicle or operating the machinery. In situations where the individual operating the vehicle or machinery is predicted to be experiencing increasing fatigue, the vehicle or machinery safety system may provide a notification or an alert to the operator of the vehicle or machinery to prompt them to take a safety action, for example, to pull off the road or to shut off the machinery.

Although the present disclosure describes methods and processes with steps in a certain order, one or more steps of the methods and processes may be omitted or altered as appropriate. One or more steps may take place in an order other than that in which they are described, as appropriate.

Although the present disclosure is described, at least in part, in terms of methods, a person of ordinary skill in the art will understand that the present disclosure is also directed to the various components for performing at least some of the aspects and features of the described methods, be it by way of hardware components, software or any combination of the two. Accordingly, the technical solution of the present disclosure may be embodied in the form of a software product. A suitable software product may be stored in a pre-recorded storage device or other similar non-volatile or non-transitory computer readable medium, including DVDs, CD-ROMs, USB flash disk, a removable hard disk, or other storage media, for example. The software product includes instructions tangibly stored thereon that enable a processing device (e.g., a personal computer, a server, or a network device) to execute examples of the methods disclosed herein. The machine-executable instructions may be in the form of code sequences, configuration information, or other data, which, when executed, cause a machine (e.g., a processor or other processing device) to perform steps in a method according to examples of the present disclosure.

The present disclosure may be embodied in other specific forms without departing from the subject matter of the claims. The described example embodiments are to be considered in all respects as being only illustrative and not restrictive. Selected features from one or more of the above-described embodiments may be combined to create alternative embodiments not explicitly described, features suitable for such combinations being understood within the scope of this disclosure.

All values and sub-ranges within disclosed ranges are also disclosed. Also, although the systems, devices and processes disclosed and shown herein may comprise a specific number of elements/components, the systems, devices and assemblies could be modified to include additional or fewer of such elements/components. For example, although any of the elements/components disclosed may be referenced as being singular, the embodiments disclosed herein could be modified to include a plurality of such elements/components. The subject matter described herein intends to cover and embrace all suitable changes in technology.

1. A method for predicting a fatigue level for a user, the method comprising:
obtaining a pair of valid photoplethysmogram (PPG) snippets from a PPG signal;
extracting a plurality of PPG features for each valid PPG snippet of the pair of valid PPG snippets;
extracting a plurality of pairwise features from the plurality of PPG features for each valid PPG snippet; and
predicting a fatigue level for the user based on the pairwise features.

2. The method of claim 1, wherein obtaining a pair of valid PPG snippets from a PPG signal comprises:
extracting a pair of raw PPG snippets from the PPG signal;
preprocessing each raw PPG snippet of the pair of raw PPG snippets to generate a pair of processed PPG snippets; and
validating each processed PPG snippet of the pair of processed PPG snippets using a trained PPG signal validator to obtain the pair of valid PPG snippets.

3. The method of claim 1, wherein extracting a plurality of PPG features for each valid PPG snippet of the pair of valid PPG snippets comprises:
detecting a plurality of peaks in each valid PPG snippet of the of the pair of valid PPG snippets;
obtaining a plurality of N-N intervals for each valid PPG snippet of the of the pair of valid PPG snippets based on the respective plurality of peaks; and
extracting a plurality of PPG features for each valid PPG snippet of the of the pair of valid PPG snippets based on the respective plurality of N-N intervals.

4. The method of claim 2, wherein preprocessing each raw PPG snippet of the pair of raw PPG snippets to generate a pair of processed PPG snippets comprises:
filtering each raw PPG snippet of the pair of raw PPG snippets with bandpass filter having a bandpass frequency 0.6-8.0 Hz; and
normalizing each raw PPG snippet of the pair of raw PPG snippets to have a mean of zero and a standard deviation of 1.

5. The method of claim 2, wherein the trained PPG signal validator is a fast fourier transform (FFT) based multi-layer perceptron network (MLP).

6. The method of claim 2, wherein the trained PPG signal validator is a temporal convolutional network (TCN).

7. The method of claim 1, wherein predicting the fatigue level comprises:
inputting the pairwise features into a progressive feature search tree to obtain a predicted fatigue level, the predicted fatigue level including an odds ratio and a probability of experiencing a change in fatigue level.

8. The method of claim 7, wherein:
prior to inputting the pairwise features into a progressive feature search tree:
building a progressive feature search tree by computing one or more relationships between a respective one or more pairwise features and a respective fatigue level.

9. The method of claim 8, wherein the one or more relationships between a respective one or more pairwise features and a respective fatigue level is computed using Fisher's exact test.

10. The method of claim 8, wherein the respective one or more pairwise features correspond to a respective tier level and the progressive feature search tree is built by progressively computing the one or more relationships between the respective one or more pairwise features and the respective fatigue level based on the respective tier level.

11. The method of claim 1, further comprising:
prior to extracting a plurality of pairwise features from the plurality of PPG features for each valid PPG snippet:
classifying the pair of valid PPG snippets using a trained differential network, the classification describing a degree of similarity between the pair of valid PPG snippets; and
in response to the pair of valid PPG snippets being classified with a low degree of similarity, extracting the plurality of pairwise features from the plurality of PPG features for each valid PPG snippet in the pair of valid PPG snippets.

12. The method of claim 1, further comprising:
comparing the fatigue level to a pre-defined criteria; and
serving a fatigue alert to the user based on the comparison.

13. A system comprising:
a PPG sensor;
one or more memories storing executable instructions; and
one or more processors coupled to the PPG sensor and one or more memories, the executable instructions configuring the one or more processors to:
obtain a pair of valid PPG snippets from a PPG signal;
extract a plurality of PPG features for each valid PPG snippet of the pair of valid PPG snippets;
extract a plurality of pairwise features from the plurality of PPG features for each valid PPG snippet; and
predict a fatigue level for the user based on the pairwise features.

14. The system of claim 13, wherein the executable instructions, when executed by the one or more processors to obtain a pair of valid PPG snippets from a PPG signal, further cause the system to:
extract a pair of raw PPG snippets from the PPG signal;
preprocess each raw PPG snippet of the pair of raw PPG snippets to generate a pair of processed PPG snippets; and
validate each processed PPG snippet of the pair of processed PPG snippets using a trained PPG signal validator to obtain the pair of valid PPG snippets.

15. The system of claim 13, wherein the executable instructions, when executed by the one or more processors to extract a plurality of PPG features for each valid PPG snippet of the pair of valid PPG snippets, further cause the system to:
detect a plurality of peaks in each valid PPG snippet of the of the pair of valid PPG snippets;
obtain a plurality of N-N intervals for each valid PPG snippet of the of the pair of valid PPG snippets based on the respective plurality of peaks; and
extract a plurality of PPG features for each valid PPG snippet of the of the pair of valid PPG snippets based on the respective plurality of N-N intervals.

16. The system of claim 13, wherein the executable instructions, when executed by the one or more processors to predict the fatigue level, further cause the system to:
input the pairwise features into a progressive feature search tree to obtain a predicted fatigue level, the predicted fatigue level including an odds ratio and a probability of experiencing a change in fatigue level.

17. The method of claim 16, wherein the executable instructions, when executed by the one or more processors, further cause the system to:
prior to inputting the pairwise features into a progressive feature search tree:
build a progressive feature search tree by computing one or more relationships between a respective one or more pairwise features and a respective fatigue level, the one or more relationships between a respective one or more pairwise features and a respective fatigue level being computed using Fisher's exact test.

18. The system of claim 13, wherein the executable instructions, when executed by the one or more processors, further cause the system to:
prior to extracting a plurality of pairwise features from the plurality of PPG features for each valid PPG snippet;
classify the pair of valid PPG snippets using a trained differential network, the classification describing a degree of similarity between the pair of valid PPG snippets; and
in response to the pair of valid PPG snippets being classified with a low degree of similarity, extract the plurality of pairwise features from the plurality of PPG features for each valid PPG snippet in the pair of valid PPG snippets.

19. The system of claim 14, wherein the executable instructions, when executed by the one or more processors, further cause the system to:
compare the fatigue level to a pre-defined criteria; and
serve a fatigue alert to the user based on the comparison.

20. A non-transitory computer-readable medium having machine-executable instructions stored thereon which, when executed by one or more processors of a computing system, cause the computing system to:
obtain a pair of valid PPG snippets from a PPG signal;
extract a plurality of PPG features for each valid PPG snippet of the pair of valid PPG snippets;
extract a plurality of pairwise features from the plurality of PPG features for each valid PPG snippet; and
predict a fatigue level for the user based on the pairwise features.
